(19) United States
(12) Patent Application Publication
(10) Pub. No.: US 2025/0275572 A1
(43) Pub. Date: Sep. 4, 2025

(54) DRIVING CIRCUIT AND POWER CONTROL FOR DRIVING PIEZOELECTRIC TRANSDUCERS

(71) Applicant: PNEUMA RESPIRATORY, INC., Boone, NC (US)

(72) Inventors: Gregory RAPP, Boone, NC (US); Shi Bo WANG, Shenzhen (CN); Jeffrey MILLER, Boone, NC (US)

(73) Assignee: PNEUMA RESPIRATORY, INC., Boone, NC (US)

(21) Appl. No.: 19/070,292

(22) Filed: Mar. 4, 2025

Related U.S. Application Data
(60) Provisional application No. 63/701,566, filed on Sep. 30, 2024.
(60) Provisional application No. 63/561,113, filed on Mar. 4, 2024.

(51) Int. Cl. A24F 40/05; A24F 40/51; A24F 40/53; A24F 40/60; A24F 40/65; A61M 15/06; H10N 30/20

(52) U.S. Cl. CPC A24F 40/05; A24F 40/51; A24F 40/53; A24F 40/60; A24F 40/65; H10N 30/20; A61M 15/06

(57) ABSTRACT

A droplet delivery device, such as for providing an aerosol for inhalation by users, includes a piezoelectric transducer that vibrates an ejector plate of an ejector mechanism. The device delivers fluid to the ejector plate that is converted to droplets that exit the device as an aerosol. A highly efficient driving circuit is provided that can precisely change the peak-to-peak voltage to the transducer and frequency while also monitoring the current from a stable voltage. Precision control while monitoring the power consumption from the piezoelectric transducer allows for consistent dosing and for the user to tailor their aerosol delivery to their preferences.

### CROSS-REFERENCE TO RELATED APPLICATIONS

This application claims the benefit of U.S. Provisional patent application Ser. No. 63/701,566 filed Sep. 30, 2024, and U.S. Provisional patent application Ser. No. 63/561,113 filed Mar. 4, 2024, which are incorporated herein by reference in their entirety.

### FIELD OF THE INVENTION

This disclosure relates to droplet delivery devices with ejector mechanisms and more specifically to droplet delivery devices for the delivery of fluids that are inhaled into mouth, throat, nose, and/or lungs.

### BACKGROUND OF THE INVENTION

The displacement of a piezoelectric transducer (piezo) is heavily dependent on the frequency and voltage of an applied waveform. For a “push mode” droplet delivery device, it is advantageous to have a highly efficient driving circuit that can precisely change the peak-to-peak voltage and frequency while monitoring the current from a stable voltage. This can be used for consistent dosing, and or user tailor of the aerosol delivery, and or targeting unique areas of the respiratory system, and the like.

In aerosol delivery, not all users are the same. This aerosol can be made of nicotine, cannabinoids, traditional Chinese medicine, any other consumer inhaled product, therapeutics, or any medicinally inhaled ingredient. Each user would likely have a preference for the delivered dose. A higher dose would also come with larger droplets. The larger droplets would cause the user to feel the aerosol more in the throat. Some users may like to not feel the aerosol at all. A piezoelectric driven droplet delivery device is capable of tailoring the spray to user preferences through the invention described in this disclosure.

In a piezoelectric driven aerosol delivery device, particle size and mass ejection are heavily dependent on the voltage and frequency applied to the piezoelectric transducer. Having precision control over these while monitoring the power consumption from the piezo allows for consistent dosing and a user to tailor their aerosol delivery to their preferences.

### SUMMARY OF THE INVENTION

The invention addresses the need for a cost-effective, efficient, and precisely adjustable method to generate a waveform for driving a piezoelectric transducer (piezo) in aerosol delivery systems. Conventional approaches lack the precision and adaptability required to meet user-specific preferences, especially in applications where customization and or consistency of aerosol output is critical. Unlike other aerosol delivery devices that prioritize basic functionality without user-specific customization, this invention achieves improved control over the voltage and frequency parameters essential for tailoring the user experience.

The circuit in invention examples is engineered to operate within a voltage range of 26 to 48 volts and at a variable frequency of about 183 kHz, with a frequency resolution finer than about 10 Hz. Moreover, the circuit achieves this precision while consuming less than 4 watts of power during actuation, showcasing its energy efficiency. By monitoring power consumption alongside precise control of voltage and frequency, the invention provides a robust solution for tailoring aerosol delivery to the specific needs of users, offering a superior level of control compared to conventional devices.

### BRIEF DESCRIPTION OF THE DRAWINGS

FIG. 1A is a relational diagram showing an overview of a circuit example implemented to create a highly efficient and effective push mode droplet delivery system. The piezoelectric driving system is shown as peripherals of the main microcontroller.

FIG. 1B is a relational diagram showing critical components of the power controlled piezoelectric driving system in an example.

FIG. 2 is a schematic diagram showing a programmable resistance circuit (113) which takes input from the microcontroller to select several different resistances for a voltage divider in an example.

FIG. 3 is a schematic diagram showing an implementation of a semiconductor amplifier (117) that can be used to drive piezoelectric devices in a push mode droplet delivery device in an example. Metal Oxide Semiconductor Field Effect Transistors (MOSFETs) (331, 333) are included in the example over Bipolar Junction Transistors (BJTs) due to greater efficiency and higher switching speeds.

FIG. 4 is a schematic diagram showing an implementation of a semiconductor amplifier (117) when the target voltage of the waveform applied to the piezoelectric transducer (119) is greater than the gate threshold voltage of the top side MOSFETs (331) added to the driving voltage of the piezoelectric transducer (301) in a example. This implementation allows the top side MOSFETs (331) to produce a high voltage waveform suitable to drive a piezoelectric transducer (119) at the same frequency as a low voltage signal input (401, 403).

FIG. 5 is a flow diagram showing a firmware algorithm carried out by the main microcontroller (100) to ensure the piezoelectric transducer (119) consumes a near-constant power during its actuation in an example.

FIG. 6A is graph showing current as measured from the current sense amplifier (105) without using the voltage regulation algorithm from FIG. 5.

FIG. 6B is a graph showing current as measured from the current sense amplifier (105) using the voltage regulation algorithm from FIG. 5.

FIG. 7A are graphs showing another method of regulating mass ejection from a droplet delivery device using pulse width modulation (PWM) on the input waveform to the semiconductor amplifier (117).

FIG. 7B are graphs showing another method similar to the method shown by FIG. 7A, with the duty cycle applied by skipping waveforms on the input waveform to the semiconductor amplifier (117).

### DETAILED DESCRIPTION

This disclosure incorporates herein by reference in their entireties the disclosures of U.S. Pat. No. 11,793,945, entitled “Droplet Delivery Device with Push Ejection,” U.S. Pat. No. 12,161,795, entitled “Small Step Size and High Resolution Aerosol Generation System and Method,” and PCT/US24/58487, entitled “Droplet Delivery Device Implementing AI.”

The displacement of a piezoelectric transducer (piezo) (119) is heavily dependent on the frequency and voltage of an applied waveform. For the push mode droplet delivery device, it is advantageous to provide a highly efficient driving circuit that can precisely change the peak-to-peak voltage and frequency while monitoring the current from a stable voltage. This can be used for consistent dosing, and or user tailor of the aerosol delivery, and or targeting unique areas of the respiratory system, and the like.

In aerosol delivery, not all users are the same. This aerosol can be made of nicotine, cannabinoids, traditional Chinese medicine, any other consumer inhaled product, therapeutics, or any medicinally inhaled ingredient. Each user would likely have a preference for the delivered dose. A higher dose would also come with larger droplets. The larger droplets would cause the user to feel the aerosol more in the throat. Some users may prefer to not feel the aerosol at all. A piezoelectric driven droplet delivery device is capable of tailoring the spray to user preferences through the invention described in this disclosure.

In a piezoelectric driven aerosol delivery device, particle size and mass ejection are heavily dependent on the voltage and frequency applied to the piezoelectric transducer (119). Having precision control over these while monitoring the power consumption from the piezo allows for consistent dosing and or the user to tailor their aerosol delivery to their preferences.

In a preferred embodiment, a droplet delivery device comprises a piezoelectric transducer (119) and a circuit configured to drive the transducer.

Examples address the need for a cost-effective, efficient, and precisely adjustable method to generate a waveform for driving a piezoelectric transducer (119) in aerosol delivery systems. Conventional approaches lack the precision and adaptability required to meet user-specific preferences, especially in applications where customization and or consistency of aerosol output is critical. Unlike other aerosol delivery devices that prioritize basic functionality without user-specific customization, this invention achieves unparalleled control over the voltage and frequency parameters essential for tailoring the user experience.

A circuit in examples is engineered to operate within a voltage range of about 26 to about 48 volts and at a variable frequency of about 183 kHz, with a frequency resolution finer than about 10 Hz. Moreover, the circuit achieves this precision while consuming less than 4 watts of power during actuation, showcasing its energy efficiency. By monitoring power consumption alongside precise control of voltage and frequency, the invention provides a robust solution for tailoring aerosol delivery to the specific needs of users, offering a superior level of control compared to conventional devices.

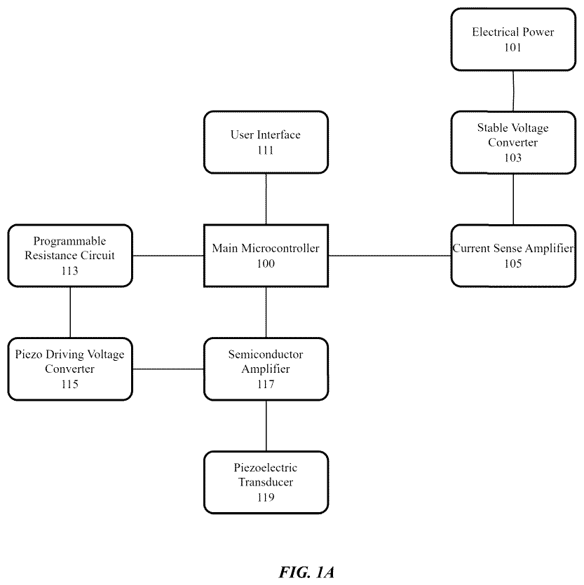

FIG. 1A and FIG. 1B show an overview of an exemplary system. The circuit is typically powered by a lithium-ion battery as an electrical power source (101) which supplies less than 5V with nominal voltage between 3.3-4.2V. The battery voltage is unfit for precise power measurements since the voltage is highly volatile during operation depending on the instantaneous current draw. The stable voltage converter (103) is a boost converter that boosts the battery voltage to a stable voltage above the battery voltage. The stable voltage is used to detect the instantaneous current draw of the piezoelectric amplifier using the current sense amplifier circuit (105). The current sense amplifier is designed to measure the current over a small magnitude (<1 ohm) shunt current sense resistor (121). The current sense amplifier (105) output is sent to the main microcontroller (100) where an analog to digital conversion (ADC) measurement is performed on the output from the current sense amplifier (105). The programmable resistance circuit (113) can be set to several different resistances depending on the input from the main microcontroller (100). The piezo driving voltage converter (115) produces a voltage depending on the output resistance of the programmable resistance circuit (113). The voltage from the piezo driving converter (115) is input into a semiconductor amplifier (117) to be used as the voltage applied to the piezoelectric transducer (119). The semiconductor amplifier (117) amplifies a waveform from the main microcontroller (100) using the voltage from the piezo driving voltage converter (115) to apply a high voltage waveform on the piezoelectric transducer (119). The user interface (111) consists of a plurality of circuits that interface with the microcontroller (100) to signal when to actuate the piezo or adjust the target power during actuation.

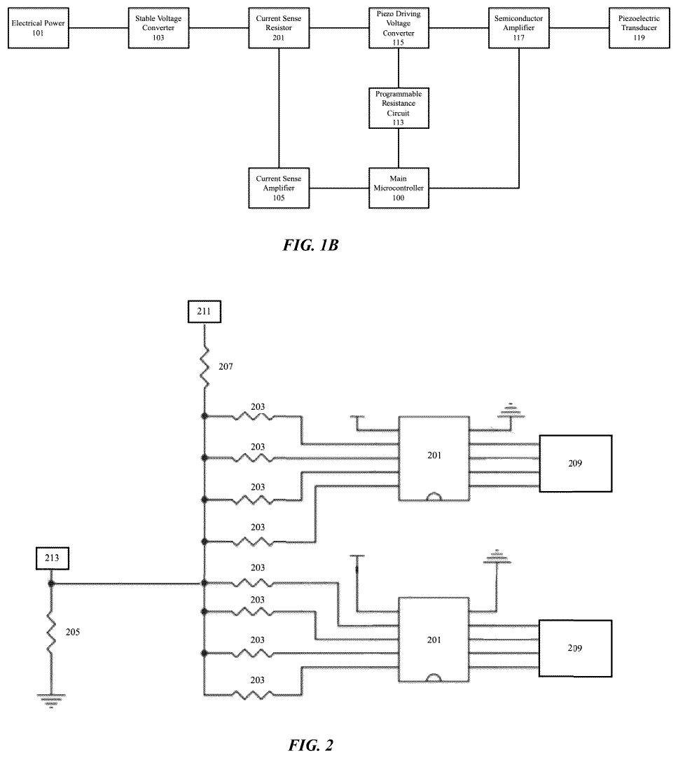

FIG. 2 shows an implementation of a programmable resistance circuit (113). In this implementation, 8 outputs from the main microcontroller (209) are wired to the inputs of two 4 input buffer chips (201). These buffer chips (201) are wired to programmable resistors (203) that are in parallel with a bottom set resistor (205) and in series with the top set resistor (207). This implementation of the programmable resistance circuit allows for 256 combinations of programmable resistor (203) selections. This circuit allows the output voltage (213) to be set digitally by the microcontroller (100) depending on the input voltage (211) and the resistance values chosen for the resistors (203, 205, 207). This circuit can be thought of as a digitally programmable voltage divider. Using this circuit on the feedback pin of the piezo driving voltage boost converter (115) allows for quick adjustments to the voltage applied to the piezoelectric transducer.

In other embodiments, more or less resistors are used to increase or decrease the number of resistor value options. In non-limiting examples, the number of resistors used can be 1, 2, 3, 4, 5, 6, 7, 8, 9, 10, 11, 12, 13, 14, 15, 16, or more.

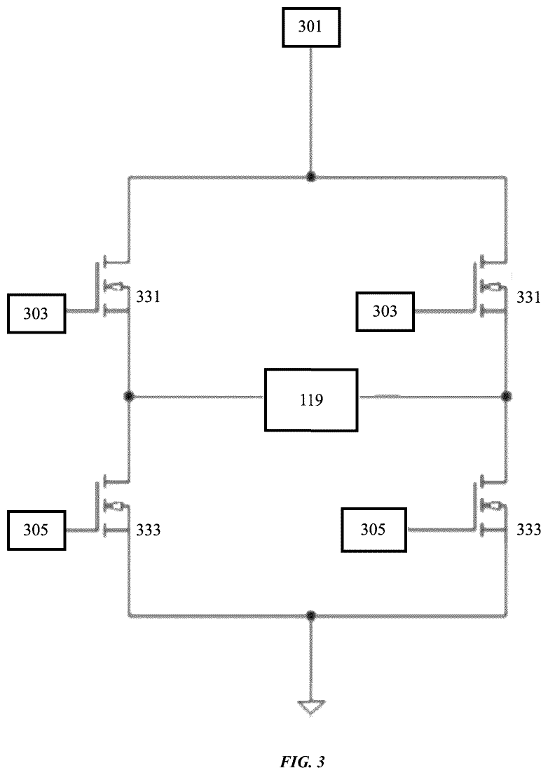

FIG. 3 shows a preferred implementation of a class AB amplifier that may be used as the semiconductor amplifier (117). MOSFETs (331, 333) are arranged in a totem pole to amplify the input waveforms (303, 305) to the same voltage as the high voltage supply (301). The goal of this circuit is to take the waveform of the main microcontroller (100) with a typical peak-to-peak voltage of around 3.3V and amplify that to the voltage of the high voltage supply (301). The top side gate voltage waveform 303 and bottom side gate voltage waveform 305 must be complementary to one another to prevent both top side MOSEFETs (331) and bottom side MOSFETs (333) from being in their conducting states at the same time to prevent a short circuit. The resulting waveform is applied to either side of the piezoelectric transducer (119) so that the apparent voltage of the piezoelectric transducer (119) is a waveform with the same frequency as the gate voltage waveforms (303, 305) and the same voltage of the high voltage supply (301).

The amplifier circuit described in FIG. 3 can be complicated to fully implement and simply tying outputs from a microcontroller (100) to the top side gate voltage waveform (303) will not function as intended. The top side input waveform (303) must be at the voltage of the high voltage supply (301) in addition to the MOSFET gate threshold voltage for the top side MOSFETs (331) to be in conductive state.

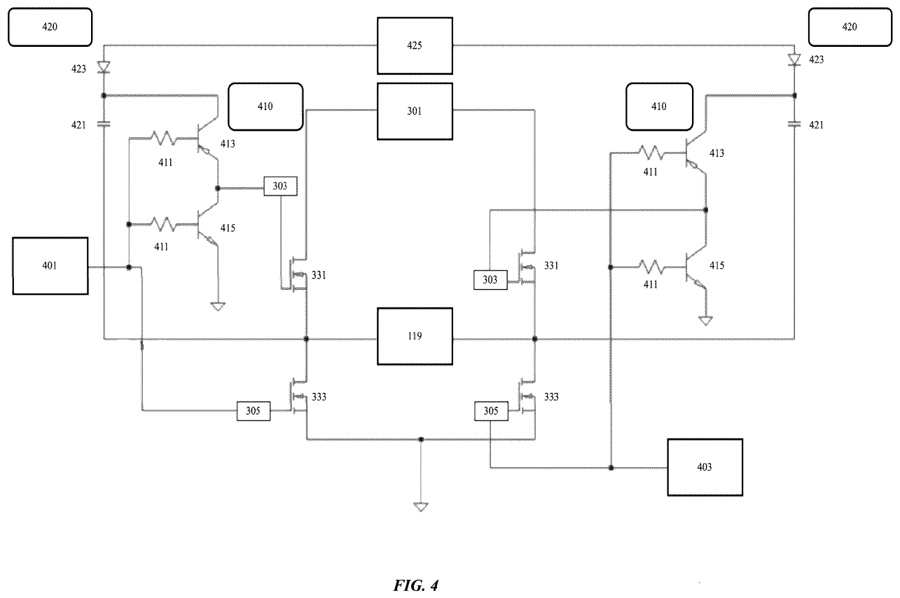

FIG. 4 shows how the boot strap circuit (420) and the totem pole circuit (410) may be necessary in order to drive a piezoelectric transducer (119) at high voltages in a MOSFET implementation of a class AB amplifier. The waveforms 401 and 403 are inputs from the main microcontroller (100) set to output the resonant frequency of the piezoelectric transducer (119). The waveforms 401 and 403 are complimentary to one another so the voltage apparent to the piezoelectric transducer is a waveform with peak-to-peak voltage value equal to twice the voltage as the high voltage supply (301). A bootstrap circuit (420) consists of a diode (423) and capacitor (421) arranged so the base of the capacitor is the output of the topside MOSFET (331) and charged by the capacitor charge voltage supply (425). This bootstrapped voltage enables a high voltage at the top side of the capacitor (421) equal to the high voltage supply (301) plus the capacitor voltage supply (425) when the top side MOSFET (331) is in its conductive state and the bottom side MOSFET (333) is in its non-conductive state. This voltage is switched onto the gate of the top side MOSFET (331) by the totem pole circuit (410). Applying the microcontroller waveforms (401, 403) to the base of the top side NPN BJT (413) and the bottom side PNP BJT (415) amplifies the frequency of the microcontroller waveform (401) onto the gate of the top side MOSFET (331). Current limiting resistors (411) are in place to limit the current to the base of the BJTs (413, 415) to appropriate base current for the BJTs. The bottom side input waveforms (305) are simpler as the bottom side MOSFET (333) source inputs are always connected to ground, so the peak-to-peak voltage can be smaller at just the MOSFET gate threshold voltage. If the proper MOSFET with a small gate threshold voltage is selected for the application, the bottom side MOSFETs may be switched using just the output from the main microcontroller (401).

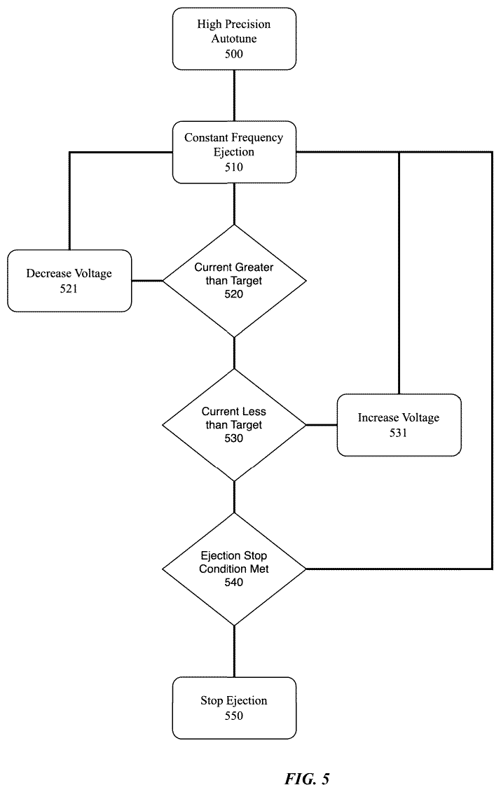

FIG. 5 describes the firmware algorithm used with the current sensing circuit (105) and the programmable resistance circuit (113) being controlled by the main microcontroller (100). A feedback circuit is described where the main microcontroller (100) will adjust the microcontroller inputs (209) to select a voltage on the piezo driving voltage converter (115) based on its instantaneous current measurements during actuation measured from the current sense amplifier (105). The purpose of this algorithm is to keep the power at a target current throughout the actuation of the piezo. The process begins with a high precision autotune (500) to find the resonant frequency of the piezoelectric transducer (119). The frequency is then static through the rest of the spray (510). During the spray, the current is monitored at a quick interval equal or less than 10 ms. If the current is greater than the target current (520) by a set threshold, the voltage is lowered (521). If the current is less than the target current (530) the voltage is increased (531). If a stop condition (540) is met based on input from the user interface peripherals (111) it will stop the ejection (550) otherwise it will continue the loop. Doing this repeatedly over the course of the ejection will result in a more stable and repeatable power consumption. This can create a more consistent piezo displacement and therefore more consistent mass ejection in our device. This algorithm also enables more accurate battery life calculations for battery powered piezoelectric transducer applications.

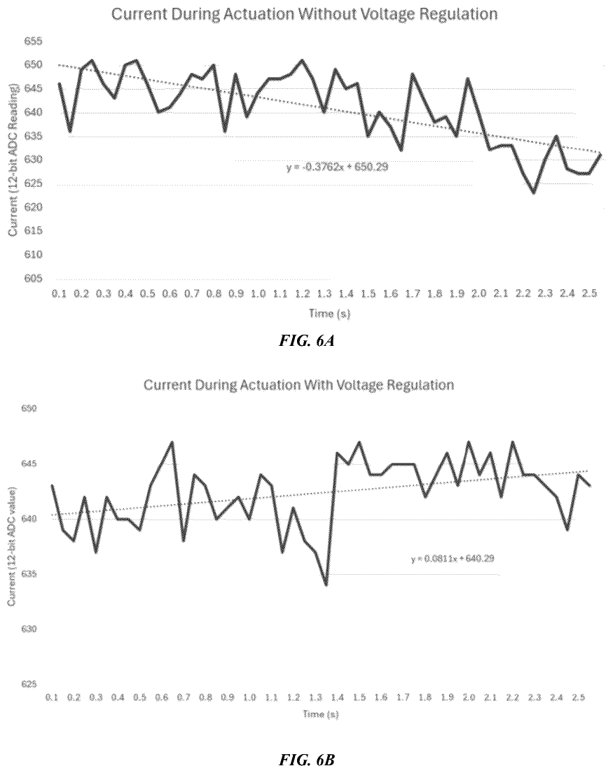

FIGS. 6A and 6B show the current during the first 2.5 seconds of actuation as recorded by the microcontroller. FIG. 6B shows how the current of the piezo without a voltage regulation algorithm tends to decrease over time when a constant frequency is applied. This is due to the piezo heating up during actuation and its resonant frequency drifting lower, as shown with the trendline. The transducer's drift in resonant frequency is increasingly apparent with increasing power applied to the transducer. Using the power regulation algorithm as described will flatten this curve to apply more consistent power to the transducer. FIG. 6B shows the resulting current while applying the algorithm described in FIG. 5. FIG. 6B shows how the current is more consistent over the course of the spray when using a power regulation algorithm.

In another embodiment, a similar power regulation algorithm could be conceived using the driving frequency of the piezoelectric. Monitoring the current and adjusting the frequency of the ejection could produce similar results to adjusting the voltage level at a constant frequency. Voltage regulation is typically preferred over frequency regulation as it has a more linear impact on the current draw from the piezoelectric transducer.

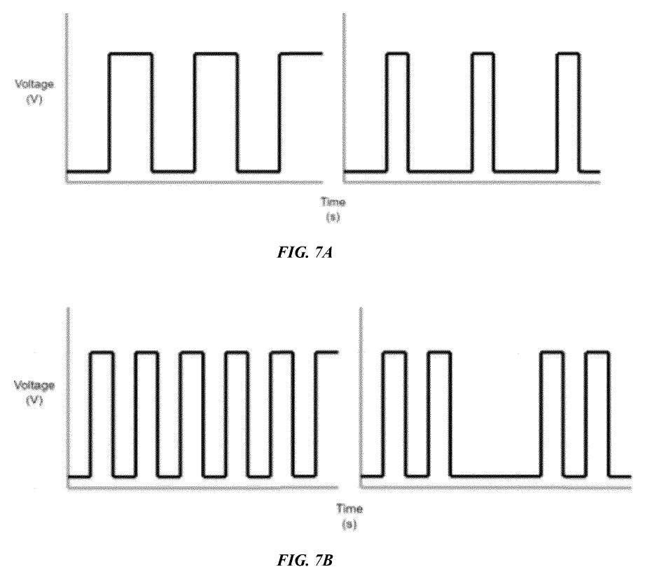

In another embodiment, pulse width modulation (PWM) can be used to change the ejection volume. PWM creates a duty cycle on the output waveform. The piezo could be on 100% of the time for a maximum amount of volume delivery. The piezo could be on 50% of the time for half of the ejection volume. This duty cycle could either be applied to every wave of the waveform in method 1 (FIG. 7A) or the entire waveform could be applied for a moment, then turned off in method 2 (FIG. 7B).

In a preferred embodiment, the frequency generated as the input to the semiconductor amplifier (401, 403) is produced by a numerically controlled oscillator by the main microcontroller (100) or a separate chip. This allows for high precision frequency adjustments of less than about 10 Hz. Possible options would be the PIC16 and PIC18 microcontrollers from Microchip or the AD9830 by Analog Devices and the like.

In another preferred embodiment, the microcontroller input waveforms (401, 403) are complimentary to one another with a brief and adjustable dead time between switching either waveform. The PIC16 and PIC18 microcontrollers have a complimentary waveform generator to produce input waveforms (401, 403) at opposite polarities through software and adjustable dead times where both outputs are set to 0V.

Controlling the ejection volume is important during normal use. As the piezoelectric transducer vibrates, it will heat up. As it heats up, the resistance of the piezo changes. This means the power delivered to the piezo will change and result in a different output. Keeping track of the current delivered to the piezo means the output will be consistent and precise.

Controlling the ejection volume also allows for the user to be able to select the ejection volume. This can be accomplished by using an application (app) on a phone or have a user control on the device itself. The user can make a selection for the ejection volume on the app, i.e., high, medium, low, or a slider bar. When the user makes a selection, the app will communicate with the microcontroller in the device via Bluetooth. The same effect of selecting a power level can be achieved via a physical user control (button, dial, etc.) placed on the device itself. The microcontroller will control the voltage output of the boost converter based on the user's input.

In an embodiment, there are various selection ranges for the user to choose from. There can be low granularity of high, medium, or low, or high granularity by giving the user the option to select up to 128 steps of varying ejection volume.

In another embodiment, the user can select the ejection volume through a physical knob. It could be a knob on the device, or a dial that subtly sticks out. The knob or dial would be connected to a potentiometer. The microcontroller can determine the resistance of the potentiometer and control the ejection volume based upon the resistance.

In a further embodiment of controlling the ejection volume. There is a pressure sensor or microphone in the device for breath actuation. If the pressure sensor or microphone determines the user is inhaling with more pressure or force, the microcontroller can be programmed to increase the ejection.

In another embodiment, a physical means of controlling the ejection volume is by tap or shakes. There can be precise vibration detectors like an accelerometer to determine taps or shakes. The button on the device can be programmed to be used to change the ejected volume.

In another embodiment, a physical means of controlling the ejection volume is through a button in the user interface (111). Manipulating multiple button presses or long button presses can change the power level, PWM, frequency delivered, or the like. Any of these will then change the ejection volume. An example of this could be the following procedure: 1) 3 second button press to enter mode to change the power level. 2) A quick button press to cycle through the ejection levels, i.e. press once to change from high to medium-high, press again to change from medium-high to medium-low, press again to change from medium-low to low, press again to cycle back to high. 3) When ejection level is selected, 3-second-long button press to enable to the level selected.

It may be advantageous to have ejection start before the user starts to inhale. This can add to the user's experience. To achieve this, the device can automatically detect when the device is moving towards a user's mouth through an accelerometer. This requires data to be recorded from the accelerometer to understand how users will move the device before inhaling. The microcontroller on the device will keep checking the accelerometer for the correct output, indicating the device is moving towards a user's mouth. The device will only eject for a maximum amount of time before turning off. This could be anywhere from 0.01 s to 3 s with an ideal time of 0.5 to 1 s. This means, the device will eject but if a user doesn't actually inhale, it doesn't continue ejecting.

In another embodiment, this could be accomplished using several other methods. One method would be by the user tapping the device to initiate the initial ejection.

In another embodiment, the button is can be pressed to create a pre-ejection. After turning the device on, the user can push the button a second time. This second button push would create a small ejection. This ejection will sit in the mouthpiece before the user inhales. The amount of ejection time can be between 0.05 s and 1 s.

In another embodiment, the button press pre-ejection occurs when the user turns on the device. This means when the user pushes the button once to awaken the device, the device does a pre-ejection into the mouthpiece for 0.05 s to 0.1 s.

In another embodiment, the device keeps a small amount of aerosol in the ejection port (the space after the ejector plate and before the tip of the mouthpiece). This would be accomplished by monitoring the movement of the device via something like an accelerometer. Additionally, it could be accomplished by monitoring the resistance of the handpiece. If a hand is in contact with the handpiece, the resistance will increase. While the device is in the hand, the device will keep aerosol in the ejection port. This will be a small ejection of somewhere between 0.01 s to 3 s with an idea time of 0.5 s to 1 s. The aerosol will dissipate after a short period of time, approximately 3 s to 60 s, at which time the device will eject again to put aerosol in the ejection port.

In another embodiment, the device would detect a hand holding the device via magnetic field monitoring, capacitive touch sensor(s), temperature sensor(s), impedance monitoring, and the like.

In another embodiment, the option to have a pre-ejection is controlled by the user. This can be controlled either using the button, a dial, or an app. The user can also control the amount of pre-ejection using the same method(s).

In another embodiment, a CO sensor can be used in the device to monitor the user's smoking habits. If a user has smoked (primarily cigarettes and potentially e-cigarettes) they will exhale carbon monoxide (CO). A CO sensor on the device will help the user track traditional smoking habits. As the user reduces traditional and potentially e-cigarette consumption, their exhaled CO will decline. This would also be great motivation for a user to visually see a metric for monitoring quitting smoking.

In another embodiment, a doctor can view the user's smoking habits from the recorded CO monitoring. The doctor can have access to the user's account in the app. Or, the user can show the doctor at their regular appointments. This will help the doctor guide the user to a healthier lifestyle.

Additionally, each ejector plate may have a slightly different ejection. In this case, the power can be changed automatically to reflect the different ejection volume. This can be achieved by having a ID chip on the ejector bracket.

Each ejector bracket has its own ejector plate. The ejector plate will be tested during the manufacturing process to determine the ejection output. Once the level is determined, the ID chip will be loaded with an identifier. The handpiece will read the identifier and set the nominal voltage level.

For example, the ejection level can be set to 75% to start. That 75% might correspond to 5 mg in ejected mass. The nominal voltage level would be the voltage to which the ejector plate will eject 5 mg of ejected mass. Then, the user can change the power output and change the ejection as needed. This will ensure that each ejector bracket will have the same ejection at each user selected power level.

In a preferred embodiment, there is an ID physically on the ejector bracket (similar to Microchip's SHA104). When the ejector bracket is inserted into the handpiece, the handpiece will physically connect to the ejector bracket to read the ID chip. The ID chip can also have information on it to provide information about the batch information such as the ejector plate manufacture date and batch as well as the ejector bracket manufacture date and batch. Also, the ID chip can have any ejector bracket/ejector plate identifying characteristics such as aperture hole size, thickness of the plate, distance between aperture holes, size of the dome, material of the ejector plate, material of the ejector bracket, the shape and material of the suspension gasket, and so on.

In another embodiment, the ejector bracket can be identified through a QR code that comes with the ejector bracket or printed on the ejector bracket. The handpiece must be connected to a mobile application for this application. The user uses their phone to scan the QR code, the information is transferred to the mobile application, and the mobile application communicates with the handpiece to set the power level.

In another embodiment, the ejector bracket can be identified through a passive element such as a resistor. A resistor can be put into the ejector bracket. When the ejector bracket is put into the handpiece, the handpiece will electrically connect to the resistor. The resistance will be read. The power level will be set based upon the resistance of the resistor. As an example, a nominal power level can be 2.00 W. If the resistor is 10 kΩ, the power stays at 2 W. If the resistor is 50 kΩ, the power increases to 2.25 W. If the resistor is 5 kΩ, the power decreases to 1.75 W.

For reference, the following elements correspond to the listed element number:

| Element Number | Element Name |
|---|---|
| 100 | Main Microcontroller |
| 101 | Electrical Power |
| 103 | Stable Voltage Converter |
| 105 | Current Sense Amplifier |
| 111 | User Interface |
| 113 | Programmable Resistance Circuit |
| 115 | Piezo Driving Voltage Converter |
| 117 | Semiconductor Amplifier |
| 119 | Piezoelectric Transducer |
| 121 | Current Sense Resistor |
| 201 | 4 input buffer |
| 203 | Programmable Resistors |
| 205 | Bottom side set resistor |
| 207 | Top side set resistor |
| 211 | Voltage divider input voltage |
| 213 | Voltage divider output voltage |
| 301 | Piezoelectric driving voltage |
| 303 | High Side Gate Voltage Waveform |
| 305 | Low Side Gate Voltage Waveform |
| 331 | Top side MOSFET |
| 333 | Bottom side MOSFET |
| 401 | Microcontroller Waveform 1 |
| 403 | Microcontroller Waveform 2 |
| 410 | Totem Pole Circuit |
| 411 | BJT base resistor |
| 413 | Top side BJT |
| 415 | Bottom side BJT |
| 420 | Boot strap Circuit |
| 421 | Boot strap diode |
| 423 | Boost strap capacitor |
| 425 | Boot strap capacitor charge voltage supply |
| 500 | High Precision Autotune |
| 510 | Constant Frequency Ejection |
| 520 | Current Greater than Target |
| 521 | Decrease Voltage |
| 530 | Current Less than Target |
| 531 | Increase Voltage |
| 540 | Ejection Stop Condition Met |
| 550 | Stop Ejection |

While described with reference to specific embodiments herein, the invention is intended to extend in scope to the full extent of the disclosure.

What is claimed:

1. A droplet delivery device comprising:
i. a piezoelectric transducer with a driving circuit including
ii. a first boost converter configured to maintain voltage stability for current measurements; and
iii. a second boost converter configured to generate high voltage for driving the piezoelectric transducer.

2. The droplet delivery device of claim 1, wherein the second boost converter generates high voltage that is in a range from about 13.5 V to about 28.9 V.

3. The droplet delivery device of claim 1, wherein the first boost converter level maintains voltage stability at about 5 V, 6 V, 7 V, 8V, 9V, 10 V, 11V, or 12 V.

4. The droplet delivery device of claim 3, wherein voltage level accuracy is in a range selected from one of 100 nV to 1 mV, 1 mV to 10 mV, 10 mV to 100 mV, and 100 mV to 1 V.

5. A droplet delivery device including a feedback circuit configured to continuously measure the current of a piezoelectric transducer and to adjust supplied voltage to keep power consistent.

6. A droplet delivery device comprising a piezoelectric transducer and a controller electronically coupled to the transducer and configured to receive control input from a user to control the amount of aerosol ejected.

7. The droplet delivery device of claim 6 wherein the control input includes controlling power controls to control the amount of aerosol ejected.

8. The droplet delivery device of claim 6 wherein the control input includes controlling pulse width modulation to control the amount of aerosol ejected.

9. The droplet delivery device of claim 6 wherein the control input to control the amount of aerosol ejected is configured to be received from a physical input directly to the device.

10. The droplet delivery device of claim 6 wherein the control input to control the amount of aerosol ejected is configured to be received from a computing device communicatively coupled to the aerosol delivery device.

11. The droplet delivery device of claim 6 wherein the control input to control the amount of aerosol ejected is configured to be received from a mobile application of the computing device.

12. A droplet delivery device comprising a microcontroller configured to create a pre-ejection of droplets before a user starts to inhale from the device.

13. The droplet delivery device of claim 12 further comprising an accelerometer, and wherein the microcontroller is programmed with an algorithm that automatically initiates the pre-ejection of droplets.

14. The droplet delivery device of claim 12 wherein the microcontroller is configured to initiate the pre-ejection of droplets following receipt of one or more user control inputs to the device.

15. The droplet delivery device of claim 6, wherein the one or more user control inputs include one or more button pushes.

16. A droplet delivery device comprising a carbon monoxide sensor configured to monitor a user's exhaled carbon monoxide.

17. The droplet delivery device of claim 16 wherein the device is communicatively coupled to a mobile application that is configured to enable a user to monitor exhaled carbon monoxide levels.

18. A droplet delivery device comprising means for boosting voltage to change the vibration levels of a piezoelectric transducer of the droplet delivery device.

19. A droplet delivery device comprising one or both of a voltage regulator and frequency regulator configured to deliver a more consistent spray based on power consumption.

20. A droplet delivery device comprising:
i. an ejector with an associated identification mechanism configured to determine operation parameters of the device; and
ii. a handpiece that can read and or communicate with the identification mechanism.

21. The droplet delivery device of claim 20, wherein the identification mechanism is an ID chip.

22. The droplet delivery device of claim 20, wherein the identification mechanism is a QR code.

23. The droplet delivery device of claim 20, wherein the identification mechanism is a passive mechanism such as a resistor.

24. The droplet delivery device of claim 20, wherein the operation parameters include nominal power level.
